(19) United States
(12) Patent Application Publication
(10) Pub. No.: US 2025/0109108 A1
(43) Pub. Date: Apr. 3, 2025

(54) COMPOUND, RUBBER BLEND CONTAINING THE COMPOUND, VEHICLE TIRE COMPRISING THE RUBBER BLEND IN AT LEAST ONE COMPONENT, PROCESS FOR PRODUCING THE COMPOUND, AND USE OF THE COMPOUND AS AN AGEING PROTECTANT AND/OR ANTIOXIDANT AND/OR DYE

(71) Applicant: Continental Reifen Deutschland GmbH, Hannover (DE)

(72) Inventors: Andreas Jacob, Stöcken (DE); David-Raphael Dauer, Stöcken (DE); Julian Strohmeier, Stöcken (DE)

(73) Assignee: Continental Reifen Deutschland GmbH, Hannover (DE)

(21) Appl. No.: 18/715,328

(22) PCT Filed: Nov. 2, 2022
(86) PCT No.: PCT/DE2022/200253

§ 371 Date: May 31, 2024

(30) Foreign Application Priority Data
Dec. 2, 2021 (DE) 10 2021 213 722.9

(51) Int. Cl. C07D 219/08; B60C 1/00; C08K 5/3437

(52) U.S. Cl. CPC C07D 219/08; B60C 1/0016; B60C 1/0025; C08K 5/3437

(57) ABSTRACT

The invention relates to a compound, to a rubber mixture containing the compound, to a vehicle tire comprising the rubber mixture in at least one component, to a process for preparing the compound and to the use of the compound as aging stabilizer and/or antioxidant and/or dye.

The compound of the invention has the following formula I): I)

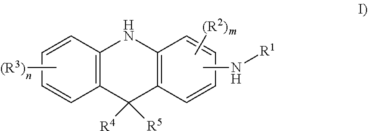

where R1 is selected from the group consisting of benzyl and linear, branched and cyclic aliphatic C3 to C12 radicals; and where the R4 and R5 radicals are independently the same or different and are each selected from the group consisting of linear, branched and cyclic aliphatic C1 to C12 radicals, and aryl radicals, cyano radicals, halogen radicals, preferably fluorine, bromine and chlorine, ester radicals, ketone radicals, ether radicals and thioether radicals, where the linear, branched and cyclic aliphatic C1 to C12 radicals and aryl radicals may bear substituents.

The present application is a National Stage Application under 35 U.S.C. § 371 of International Patent Application No. PCT/DE2022/200253 filed on Nov. 2, 2022, and claims priority from German Patent Application No. 10 2021 213 722.9 filed on Dec. 2, 2021, in the German Patent and Trademark Office, the disclosures of which are herein incorporated by reference in their entireties.

The invention relates to a compound, to a rubber mixture containing the compound, to a vehicle tire comprising the rubber mixture in at least one component, to a process for preparing the compound and to the use of the compound as aging stabilizer and/or antioxidant and/or dye.

### BRIEF SUMMARY

It is known that polymeric materials such as, in particular, rubbers are used in vehicle tires and industrial rubber articles.

Natural rubber and synthetic polymers (such as IR, BR, SBR, ESBR etc.), but also natural and synthetic oils, fats and lubricants, in the event of case of prolonged storage and in particular in the target application which is often at elevated temperatures, are subject to oxidation reactions which have an adverse effect on the originally desired properties. Depending on the type of the polymer, the polymer chains are shortened right up to liquefaction of the material, or the material subsequently hardens.

Aging stabilizers thus contribute to a crucial degree to the service life of vehicle tires and other industrial rubber articles.

Known aging stabilizers are aromatic amines, for example
6PPD (N-(1,3-dimethylbutyl)-N′-phenyl-p-phenylenediamine),
IPPD (N-isopropyl-N′-phenyl-p-phenylenediamine) or
SPPD (N-(1-phenylethyl)-N′-phenyl-p-phenylenediamine).

These molecules can react with oxygen or ozone or free radicals formed, such as alkyl, alkoxy and alkylperoxy radicals, and thus scavenge these and accordingly protect the rubbers etc. from further oxidation reactions.

However, a disadvantage of this substance class is that they could be carcinogenic.

Aging stabilizers which especially react with and scavenge ozone are also referred to as “antiozonants”.

It is an object of the invention to provide a novel compound which can especially be used as aging stabilizer in vehicle tires or other industrial rubber articles, specifically with a lower hazard potential coupled with sufficient solubility in the respective matrix, for example and in particular in the polymer. This is intended to ensure continuing optimal protection from oxygen and ozone and to prevent the tendency for blooming while reducing hazardousness to health.

### DETAILED DESCRIPTION

The object is achieved by the inventive compound as claimed in claim 1, by the inventive rubber mixture containing the compound, and by the inventive vehicle tire comprising the inventive rubber mixture in at least one component.

The object is also achieved by the process of the invention for preparing the compound of the invention.

The compound as claimed in claim 1 has the general formula I):

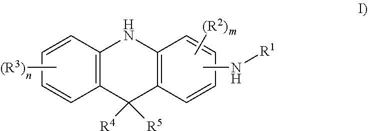

where R¹ is selected from the group consisting of benzyl and linear, branched and cyclic aliphatic C₃ to C₁₂ radicals; and
where R³ is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, and where n assumes the value of 0 or 1 or 2 or 3 or 4, where the R³ radicals, in the case that n=2 or 3 or 4, are independently the same or different, and
where R² is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals; and
where m assumes the value of 0 or 1 or 2 or 3, where the R² radicals, in the case that m=2 or 3, are independently the same or different, and
where the R⁴ and R⁵ radicals are independently the same or different and are each selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, where the linear, branched and cyclic aliphatic C₁ to C₁₂ radicals and aryl radicals may bear substituents.

It is apparent to those skilled in the art that n is 0 (zero) or 1 or 2 or 3, a hydrogen atom is bonded in each case to the corresponding carbon atom of the benzene ring rather than R³. Likewise, in the case that m is 0 or 1 or 2, all other clear positions on the benzene ring of the skeleton are hydrogen atoms.

It will likewise be clear to those skilled in the art that the representation of the linkages of (R²)ₘ and (R³)ₙ and R¹HN to the respective benzene ring of the skeleton means that these groups may each be disposed at any position on the respective benzene ring, except of course for two or more simultaneously at the same position, which would be ruled out merely by the tetravalency of the carbon atom of the benzene ring.

In the context of the present invention, expressions such as “C₃ to C₁₂ radicals” mean radicals having 3 to 12 carbon atoms. Irrespective of this, “C₁” is also used to describe the position of the most highly oxidized carbon atom/the highest priority according to the Cahn-Ingold-Prelog convention (CIP). It will be clear to the person skilled in the art what is meant in the respective context.

The compound of the invention is an acridine derivative and exhibits a lower hazard potential compared to known aging stabilizers based on aniline (possible cleavage product of 6PPD).

The compound of the invention, in rubber mixtures, shows a comparable or even improved anti-aging effect compared to 6PPD and is thus suitable as a substitute for 6PPD, the degradation products of which are extremely toxic to the silver salmon and hence probably also to other aquatic organisms.

The compound of the invention additionally has very good solubility in rubber mixtures, especially for vehicle tires and other industrial rubber articles. This prevents bleeding of this compound, as known from other aging stabilizers.

The invention encompasses all the advantageous embodiments reflected in the claims inter alia. The invention especially also encompasses embodiments which result from a combination of different features with different levels of preference for these features, and so the invention also encompasses a combination of a first feature described as “preferred” or described in the context of an advantageous embodiment with a further feature described for example as “particularly preferred”.

The inventive compound of formula I) is particularly suitable as aging stabilizer and/or antiozonant in vehicle tires and/or other industrial rubber articles, such as in particular an air spring, a bellows, conveyor belt, strap, drive belt, hose, rubber band, profile, a seal, a membrane, tactile sensors for medical applications or robotics applications, or a shoe sole or parts thereof, and/or oils and/or lubricants.

The inventive compound of formula I) is particularly suitable for producing a rubber article, in particular an air spring, a bellows, conveyor belt, strap, drive belt, hose, rubber band, profile, a seal, a membrane, tactile sensors for medical applications or robotics applications, or a shoe sole or parts thereof.

For use of the compound according to formula I) in the recited articles or substances, said compound is used in a composition and used incorporated in said composition.

In vehicle tires or other industrial rubber articles, said composition is in particular a rubber mixture.

The invention further provides for the use of the inventive compound of formula I) in oils and lubricants, such as in particular fuels or operating media for engines. In particular, the compound of the invention may must be used in engines.

The invention further provides for the use of the inventive compound of formula I) as dye in fibers and/or polymers and/or paper and/or in (decorating) paints and coatings.

Preferably, in formula I), n is 0 (zero).

Preferably, in formula I), m is 0 (zero).

It is preferable that R¹ is bonded to the nitrogen atom (N) via a tertiary carbon atom. This means that the C₁ atom is preferably a tertiary carbon atom.

In the context of the present invention, the term “tertiary carbon atom” shall be understood to mean a carbon atom bonded to only one hydrogen atom.

This results in particular good solubility of the compound of the invention in rubber mixtures, especially for vehicle tires and other industrial rubber articles, and optimize the activity in association with the mechanisms of relevance for aging stabilization.

More preferably, R¹ is a branched alkyl radical having 3 to 12 carbon atoms, preferably in turn 3 to 8 carbon atoms. In this case, there is preferably at least one branch at the C₁ atom, i.e. at the carbon atom bonded to the nitrogen atom (N), which means that the C₁ atom is a tertiary carbon atom.

This results in particular good solubility of the compound of the invention in rubber mixtures, especially for vehicle tires and other industrial rubber articles, and optimize the activity in association with the mechanisms of relevance for aging stabilization.

Even more preferably, R¹ is selected from 1,3-dimethylbutyl and cyclohexyl radicals; even more preferably in turn, R¹ is a 1,3-dimethylbutyl radical.

This results in a particularly good solubility of the compound of the invention in rubber mixtures, in particular for vehicle tires and other industrial rubber articles.

The linear, branched and cyclic aliphatic C₁ to C₁₂ radicals and aryl radicals may bear substituents.

Preferably, the R⁴ and R⁵ radicals are each selected from the group consisting of unsubstituted linear, branched and cyclic aliphatic C₁ to C₁₂ radicals, and aryl radicals. More preferably, the R⁴ and R⁵ radicals here are each selected from the group consisting of methyl radicals, n-butyl radicals and phenyl radicals.

These radicals are particularly readily obtainable via synthesis proceeding from the corresponding lithium compounds.

Most preferably, the R⁴ and R⁵ radicals are each selected from the group consisting of linear C₁ to C₁₂ radicals, more preferably consisting of linear C₁ to C₆ radicals, most preferably methyl radicals.

This results in particular good solubility of the compound of the invention in rubber mixtures, especially for vehicle tires and other industrial rubber articles, and optimize the activity in association with the mechanisms of relevance for aging stabilization.

Moreover, the compound of the invention thus has very good stability, especially with respect to oxidation to acridine.

In particularly preferred embodiments of the invention, the R⁴ and R⁵ radicals are the same.

Most preferably, each of R⁴ and R⁵ is a methyl radical.

This results in optimal solubility of the compound of the invention in rubber mixtures, in particular for vehicle tires and other industrial rubber articles.

Moreover, the compound of the invention thus has very good stability, especially with respect to oxidation to acridine.

In a preferred embodiment, the inventive compound of formula I) has the structure of formula II):

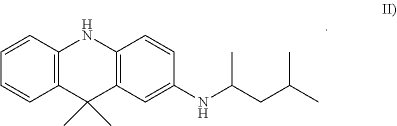 [SMILES: CC(C)CC(C)Nc1ccc2c(c1)C(C)(C)c1ccccc1N2]

The compound of formula II) achieves the object underlying the invention particularly efficiently.

The compound of formula II) actually makes it possible to achieve a further improvement in protection against oxidation and thus aging, in particular in polymers. At the same time, the compound of formula II) is markedly less hazardous to health than, for example, 6PPD or other representatives of this substance class, as mentioned by way of introduction.

Compared to 6PPD, the compound of formula II) is therefore a better aging stabilizer which is simultaneously less hazardous to health and more environmentally friendly.

In further preferred embodiments, the R⁴ and R⁵ radicals are different. More preferably, in this case, one of the R⁴ and R⁵ radicals is an aryl radical, especially a phenyl radical, and the other is selected from the group consisting of linear C₁ to C₁₂ radicals, more preferably linear C₁ to C₆ radicals, and most preferably a methyl radical.

In a further preferred embodiment, the inventive compound of formula I) has the structure of formula III):

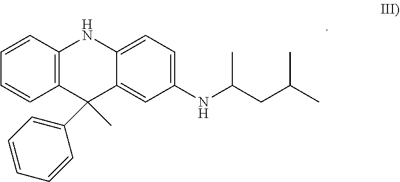 [SMILES: CC(C)CC(C)Nc1ccc2c(c1)C(C)(c1ccccc1)c1ccccc1N2]

As mentioned above, the invention further provides a rubber mixture.

The rubber mixture of the invention contains the compound of formula I). The rubber mixture of the invention may in principle be any rubber mixture in which in particular the novel inventive compound of formula I) acts as an aging stabilizer and/or antiozonant with low toxicity.

The rubber mixture of the invention contains at least one rubber.

It is preferable when the rubber mixture of the invention contains 0.1 to 10 phr, more preferably 0.1 to 7 phr, even more preferably 1 to 6 phr, of the compound of formula I).

The unit “phr” (parts per hundred parts of rubber by weight) used in this document is the conventional indication of quantity for mixture recipes in the rubber industry. The dosage of the parts by weight of the individual substances is based in this document on 100 parts by weight of the total mass of all high molecular weight (M_{w} greater than 20 000 g/mol) rubbers present in the mixture.

In advantageous embodiments of the invention, the rubber mixture of the invention contains at least one diene rubber.

The rubber mixture may accordingly contain a diene rubber or a mixture of two or more different diene rubbers.

Diene rubbers are rubbers which are formed by polymerization or copolymerization of dienes and/or cycloalkenes and thus have C═C double bonds either in the main chain or in the side groups.

The diene rubber is preferably selected from the group consisting of natural polyisoprene (NR), synthetic polyisoprene (IR), epoxidized polyisoprene (ENR), butadiene rubber (BR), butadiene-isoprene rubber, solution-polymerized styrene-butadiene rubber (SSBR), emulsion-polymerized styrene-butadiene rubber (ESBR), styrene-isoprene rubber, liquid rubbers having a molecular weight M_{w} of more than 20 000 g/mol, halobutyl rubber, polynorbornene, isoprene-isobutylene copolymer, ethylene-propylene-diene rubber, nitrile rubber, chloroprene rubber, acrylate rubber, fluoro rubber, silicone rubber, polysulfide rubber, epichlorohydrin rubber, styrene-isoprene-butadiene terpolymer, hydrogenated acrylonitrile butadiene rubber and hydrogenated styrene-butadiene rubber.

Nitrile rubber, hydrogenated acrylonitrile-butadiene rubber, chloroprene rubber, butyl rubber, halobutyl rubber and/or ethylene-propylene-diene rubber in particular are used in the production of technical rubber articles, such as belts, drive belts and hoses, and/or shoe soles. The mixture compositions known to those skilled in the art for these rubbers, which are specific in terms of fillers, plasticizers, vulcanization systems and additives, are preferably used here.

The natural and/or synthetic polyisoprene of all embodiments may be either cis-1,4-polyisoprene or 3,4-polyisoprene. However, the use of cis-1,4-polyisoprenes having a cis-1,4 proportion of >90% by weight is preferred. Such a polyisoprene is firstly obtainable by stereospecific polymerization in solution with Ziegler-Natta catalysts or using finely divided lithium alkyls. Secondly, natural rubber (NR) is one such cis-1,4-polyisoprene, for which the cis-1,4 content in the natural rubber is greater than 99% by weight.

A mixture of one or more natural polyisoprenes with one or more synthetic polyisoprenes is further also conceivable.

In the context of the present invention, the term “natural rubber” means naturally occurring rubber which may be obtained from Hevea rubber trees and from “non-Hevea” sources. Non-Hevea sources include for example guayule shrubs and dandelion such as for example TKS (Taraxacum kok-saghyz; Russian dandelion).

If the rubber mixture of the invention contains butadiene rubber (i.e. BR, polybutadiene), this may be any of the types known to those skilled in the art. These include what are called the high-cis and low-cis types, with polybutadiene having a cis content of not less than 90% by weight being referred to as the high-cis type and polybutadiene having a cis content of less than 90% by weight being referred to as the low-cis type. An example of a low-cis polybutadiene is Li—BR (lithium-catalyzed butadiene rubber) having a cis content of 20% to 50% by weight. Particularly good properties and low hysteresis of the rubber mixture are achieved with a high-cis BR.

The polybutadiene(s) used may be end group-modified with modifications and functionalizations and/or be functionalized along the polymer chains. The modification may be selected from modifications with hydroxyl groups and/or ethoxy groups and/or epoxy groups and/or siloxane groups and/or amino groups and/or aminosiloxane and/or carboxyl groups and/or phthalocyanine groups and/or silane-sulfide groups. However, further modifications known to those skilled in the art, also referred to as functionalizations, are also useful. Metal atoms may be a constituent of such functionalizations.

In the case where at least one styrene-butadiene rubber (styrene-butadiene copolymer) is present in the rubber mixture, this may be solution-polymerized styrene-butadiene rubber (SSBR) and emulsion-polymerized styrene-butadiene rubber (ESBR), and it is also possible to use a mixture of at least one SSBR and at least one ESBR. The terms “styrene-butadiene rubber” and “styrene-butadiene copolymer” are used synonymously in the context of the present invention.

The styrene-butadiene copolymer used may be end group-modified and/or functionalized along the polymer chains with the modifications and functionalizations recited above for the polybutadiene.

The at least one diene rubber is preferably selected from the group consisting of natural polyisoprene (NR, natural rubber), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

In a particularly preferred embodiment of the invention, the at least one diene rubber is selected from the group consisting of natural polyisoprene (NR), synthetic polyisoprene (IR), butadiene rubber (BR), solution-polymerized styrene-butadiene rubber (SSBR) and emulsion-polymerized styrene-butadiene rubber (ESBR).

In a particularly advantageous embodiment of the invention, the rubber mixture comprises at least one natural polyisoprene (NR), preferably in amounts of 50 to 100 phr, and in one particularly advantageous embodiment of the invention 80 to 100 phr, even more preferably 95 to 100 phr, preferably in turn 100 phr. Such a rubber mixture especially shows optimized tear properties and abrasion properties coupled with good processibility and reversion stability.

If the rubber mixture contains less than 100 phr of NR, it preferably contains, as a further rubber, at least one diene rubber selected from the group consisting of synthetic polyisoprene (IR), butadiene rubber (BR), solution-polymerized styrene-butadiene rubber (SSBR) and emulsion-polymerized styrene-butadiene rubber (ESBR).

In a further particularly advantageous embodiment of the invention, the rubber mixture comprises at least one natural polyisoprene (NR), preferably in amounts of 5 to 55 phr, and in one particularly advantageous embodiment of the invention 5 to 25 phr, even more preferably 5 to 20 phr. Such a rubber mixture especially exhibits good processibility and reversion stability and optimized tear properties and optimal rolling resistance characteristics.

In a further particularly advantageous embodiment of the invention, the rubber mixture comprises at least one polybutadiene (BR, butadiene rubber), preferably in amounts of 10 to 80 phr, more preferably 10 to 50 phr, and in a particularly advantageous embodiment of the invention 15 to 40 phr. This achieves particularly good tear and abrasion properties of the rubber mixture of the invention and optimal braking characteristics.

In a further particularly advantageous embodiment of the invention, the rubber mixture comprises at least one solution-polymerized styrene-butadiene rubber (SSBR), preferably in amounts of 10 to 80 phr, more preferably 30 to 80 phr, and in one particularly advantageous embodiment of the invention 50 to 70 phr. This achieves particularly good rolling resistance properties of the rubber mixture of the invention. In particularly advantageous embodiments of the invention, SSBR is used in combination with at least one further rubber to achieve an optimal and balanced profile of properties.

It is preferable when the rubber mixture contains at least one filler, preferably in amounts of 30 to 500 phr, more preferably 50 to 400 phr, in turn preferably 80 to 300 phr.

In advantageous embodiments of the invention, the filler is a reinforcing filler which is preferably selected from the group consisting of carbon blacks and silicon dioxide.

Suitable carbon blacks include any carbon black types known to those skilled in the art. It is preferable when the carbon black is selected from industrial carbon blacks and pyrolysis carbon blacks, wherein industrial carbon blacks are more preferred.

It is preferable when the carbon black has an iodine number according to ASTM D 1510, also known as the iodine adsorption number, between 30 and 250 g/kg, preferably 30 to 180 g/kg, more preferably 40 to 180 g/kg, and even more preferably 40 to 130 g/kg, and a DBP number according to ASTM D 2414 of 30 to 200 ml/100 g, preferably 70 to 200 ml/100 g, more preferably 90 to 200 ml/100 g.

The DBP number in accordance with ASTM D 2414 determines the specific absorption volume of a carbon black or a light-colored filler by means of dibutyl phthalate.

The use of such a type of carbon black in the rubber mixture, in particular for vehicle tires, ensures the best possible compromise between abrasion resistance and heat buildup, which in turn influences the ecologically relevant rolling resistance.

A particularly suitable and preferred carbon black is one having an iodine adsorption number between 80 and 110 g/kg and a DBP number of 100 to 130 ml/100 g, such as in particular carbon black of the N339 type.

The silicon dioxide is preferably amorphous silicon dioxide, for example precipitated silica, which is also referred to as precipitated silicon dioxide. However, it is alternatively also possible to use fumed silicon dioxide for example.

However, particular preference is given to using a finely divided, precipitated silica which has a nitrogen surface area (BET surface area) (according to DIN ISO 9277 and DIN 66132) of 35 to 400 m²/g, preferably of 35 to 350 m²/g, more preferably of 85 to 320 m²/g and even more preferably of 120 to 235 m²/g and a CTAB surface area (according to ASTM D 3765) of 30 to 400 m²/g, preferably of 30 to 330 m²/g, more preferably of 80 to 300 m²/g and even more preferably of 115 to 200 m²/g. Such silicas lead, for example in rubber mixtures for tire treads, to particularly good physical properties of the vulcanizates. Advantages in mixture processing by way of a reduction in mixing time can also arise here while retaining the same product properties, leading to improved productivity. Silicas used may thus, for example, be either those of the Ultrasil® VN3 type (trade name) from Evonik or highly dispersible silicas known as HD silicas (e.g. Zeosil® 1165 MP from Solvay).

In particularly advantageous embodiments of the invention, the rubber mixture contains at least one silica as filler, preferably in amounts of 30 to 500 phr, more preferably 50 to 400 phr, in turn preferably 80 to 300 phr.

In these quantities, silica is especially present as the sole or primary filler (more than 50% by weight based on total filler amount).

In further advantageous embodiments of the invention, the rubber mixture contains at least one silica as further filler, preferably in amounts of 5 to 100 phr, more preferably 5 to 80 phr, in turn preferably 10 to 60 phr.

In these quantities, silica is especially present as a further filler in addition to another primary filler, such as in particular a carbon black.

The terms “silicic acid” and “silica” are used synonymously in the context of the present invention.

In particularly advantageous embodiments of the invention, the rubber mixture of the invention contains 0.1 to 60 phr, preferably 3 to 40 phr, more preferably 5 to 30 phr, even more preferably 5 to 15 phr, of at least one carbon black. In these quantities, carbon black is especially present as a further filler in addition to a primary filler, such as in particular silica.

In further advantageous embodiments of the invention, the rubber mixture of the invention contains 30 to 300 phr, preferably 30 to 200 phr, more preferably 40 to 100 phr, of at least one carbon black. In these quantities, carbon black is present as the sole or primary filler and is optionally present in combination with silica in the abovementioned smaller amounts.

In a particularly advantageous embodiment of the invention, the rubber mixture contains 5 to 60 phr, more preferably 5 to 40 phr, of at least one carbon black and 50 to 300 phr, preferably 80 to 200 phr, of at least one silica.

The rubber mixture may additionally contain further fillers which are reinforcing or non-reinforcing.

Within the context of the present invention, the further (non-reinforcing) fillers include aluminosilicates, kaolin, chalk, starch, magnesium oxide, titanium dioxide, or rubber gels and also fibers (for example aramid fibers, glass fibers, carbon fibers, cellulose fibers).

Further optionally reinforcing fillers are, for example, carbon nanotubes (CNT), including discrete CNTs, so-called hollow carbon fibres (HCF) and modified CNTs containing one or more functional groups, such as hydroxyl, carboxyl and carbonyl groups), graphite and graphene, and so-called “carbon-silica dual-phase fillers”.

In the context of the present invention zinc oxide is not included among the fillers.

The rubber mixture may further contain customary additives in customary parts by weight which are added preferably in at least one primary mixing stage during the production of said mixture. These additives include
a) aging stabilizers known in the prior art, for example p-phenylenediamine, such as N-phenyl-N′-(1,3-dimethylbutyl)-p-phenylenediamine (6PPD), N,N′-diphenyl-p-phenylenediamine (DPPD), N-(1-phenylethyl)-N′-phenyl-p-phenylenediamine (SPPD), N,N′-ditolyl-p-phenylenediamine (DTPD), N-(1,4-dimethylpentyl)-N′-phenyl-p-phenylenediamine (7PPD), N-isopropyl-N′-phenyl-p-phenylenediamine (IPPD),
or dihydroquinolines, such as 2,2,4-trimethyl-1,2-dihydroquinoline (TMQ),
b) activators, for example zinc oxide and fatty acids (e.g. stearic acid) and/or other activators, such as zinc complexes, for example zinc ethylhexanoate,
c) activators and/or agents for binding fillers, in particular carbon black or silica, for example S-(3-aminopropyl)thiosulfuric acid and/or metal salts thereof (bonding of carbon black) and silane coupling agents (binding to silicon dioxide, in particular silica),
d) antiozonant waxes,
e) resins, especially tackifying resins,
f) masticating aids, for example 2,2′-dibenzamidodiphenyl disulfide (DBD), and
g) processing aids, such as in particular fatty acid esters and metal soaps, for example zinc soaps and/or calcium soaps,
h) plasticizers, such as in particular aromatic, naphthenic or paraffinic mineral oil plasticizers, for example MES (mild extraction solvate) or RAE (residual aromatic extract) or TDAE (treated distillate aromatic extract), or rubber-to-liquid oils (RTL) or biomass-to-liquid oils (BTL) preferably having a content of polycyclic aromatics of less than 3% by weight according to method IP 346 or triglycerides, for example rapeseed oil or factices or hydrocarbon resins or liquid polymers having an average molecular weight (determination by GPC=gel permeation chromatography, in accordance with BS ISO 11344:2004) between 500 and 20 000 g/mol.

When using mineral oil this is preferably selected from the group consisting of DAE (distillate aromatic extracts), RAE (residual aromatic extract), TDAE (treated distillate aromatic extracts), MES (mild extracted solvents) and naphthenic oils.

In particularly advantageous embodiments the rubber mixture according to the invention does not contain any aging stabilizers from the group of p-phenylenediamines, in particular those listed above under a), alongside the inventive compound of formula I). In a particularly preferred embodiment the rubber mixture of the invention especially contains 0 to 0.1 phr, in particular 0 phr, of further aging stabilizers based on p-phenylenediamines and selected from the group comprising, preferably consisting of, N-phenyl-N′-(1,3-dimethylbutyl)-p-phenylenediamine (6PPD), N-(1-phenylethyl)-N′-phenyl-p-phenylenediamine (SPPD), N,N′-diphenyl-p-phenylenediamine (DPPD), N,N′-ditolyl-p-phenylenediamine (DTPD), N-isopropyl-N′-phenyl-p-phenylenediamine (IPPD), N-(1,4-dimethylpentyl)-N′-phenyl-p-phenylenediamine (7PPD).

With the preferably very small amounts of 0 to 0.1 phr, more preferably 0 phr, of p-phenylenediamines, and of the compound of formula I) present in accordance with the invention, it is possible to achieve a comparable protective effect with lower toxicity. The inventive compound of formula I) here replaces the p-phenylenediamines mentioned that are known in the art.

In further advantageous embodiments of the invention, at least one further representative of the recited p-phenylenediamine aging stabilizers is present, and so the compound of the invention only partially replaces the p-phenylenediamines known in the prior art. This also achieves the advantage of the invention, just not to an optimal extent.

In advantageous embodiments aging stabilizers based on dihydroquinoline, such as TMQ, are present in the rubber mixture in addition to the inventive compound of formula I). The amount of dihydroquinolines present, such as especially TMQ, is preferably 0.1 to 3, in particular 0.5 to 1.5, phr.

Antiozonant waxes (group d above) are considered separately and in preferred embodiments of the invention are present in the rubber mixture irrespective of whether additional aging stabilizers a) are present.

The silane coupling agents may be any of the types known to those skilled in the art.

Furthermore, one or more different silane coupling agents may be used in combination with one another. The rubber mixture may thus contain a mixture of different silanes.

The silane coupling agents react with the surface silanol groups of the silicon dioxide, in particular of the silica, or other polar groups during the mixing of the rubber/the rubber mixture (in situ) or in the context of a pretreatment (premodification) even before addition of the filler to the rubber.

Coupling agents known from the prior art are bifunctional organosilanes having at least one alkoxy, cycloalkoxy or phenoxy group as a leaving group on the silicon atom and having as another functionality a group which, possibly after cleavage, can enter into a chemical reaction with the double bonds of the polymer. The latter group may for example comprise the following chemical groups: —SCN, —SH, —NH₂ or —Sₓ— (with x=2 to 8).

Employable silane coupling agents thus include for example 3-mercaptopropyltriethoxysilane, 3-thiocyanatopropyltrimethoxysilane or 3,3′-bis(triethoxysilylpropyl) polysulfides having 2 to 8 sulfur atoms, for example 3,3′-bis(triethoxysilylpropyl) tetrasulfide (TESPT), the corresponding disulfide (TESPD), or else mixtures of the sulfides having 1 to 8 sulfur atoms with different contents of the various sulfides. TESPT may for example also be added as a mixture with industrial carbon black (trade name X50S® from Evonik).

Blocked mercaptosilanes as known for example from WO 99/09036 may also be used as a silane coupling agent. It is also possible to use silanes as described in WO 2008/083241 A1, WO 2008/083242 A1, WO 2008/083243 A1 and WO 2008/083244 A1. Usable silanes include, for example, those sold by Momentive, USA in a number of variants under the NXT name, such as especially 3-octanoylthio-1-propyltriethoxysilane, or those sold by Evonik Industries under the VP Si 363® name.

The total proportion of further additives is preferably 3 to 150 phr, more preferably 3 to 100 phr and most preferably 5 to 80 phr.

Zinc oxide (ZnO) may be included in the total proportion of further additives in the abovementioned amounts.

This may be any type of zinc oxide known to those skilled in the art, for example ZnO granules or powder. The zinc oxide conventionally used generally has a BET surface area of less than 10 m²/g. However, it is also possible to use a zinc oxide having a BET surface area of 10 to 100 m²/g, for example “nano zinc oxides”.

The rubber mixture of the invention is preferably used in vulcanized form, in particular in vehicle tires or other vulcanized industrial rubber articles.

The terms “vulcanized” and “crosslinked” are used synonymously in the context of the present invention.

The vulcanization of the rubber mixture of the invention is preferably conducted in the presence of sulfur and/or sulfur donors with the aid of vulcanization accelerators, it being possible for some vulcanization accelerators to act simultaneously as sulfur donors. The accelerator is selected from the group consisting of thiazole accelerators, mercapto accelerators, sulfenamide accelerators, thiocarbamate accelerators, thiuram accelerators, thiophosphate accelerators, thiourea accelerators, xanthogenate accelerators and guanidine accelerators.

It is preferable to use a sulfenamide accelerator selected from the group consisting of N-cyclohexyl-2-benzothiazolesulfenamide (CBS), N,N-dicyclohexylbenzothiazole-2-sulfenamide (DCBS), benzothiazyl-2-sulfenomorpholide (MBS), N-tert-butyl-2-benzothiazylsulfenamide (TBBS) and guanidine accelerators such as diphenylguanidine (DPG).

The sulfur donor substance used may be any sulfur donor substances known to those skilled in the art.

Vulcanization retarders may also be present in the rubber mixture.

The production of the rubber mixture is preferably otherwise effected by the process customary in the rubber industry in which a primary mixture comprising all constituents except the vulcanization system (for example sulfur and vulcanization-influencing substances) is first produced in one or more mixing stages. The finished mixture is produced by adding the vulcanization system in a final mixing stage.

The finished mixture is for example processed further and brought into the appropriate shape by means of an extrusion operation or calendering.

The rubber mixture of the invention is particularly suitable for use in vehicle tires, especially pneumatic vehicle tires. Use in all tire components is conceivable in principle, in particular in an outer component, in particular and preferably in the flange profile, tread and/or sidewall. In the case of a tread with cap/base construction, the rubber mixture of the invention is preferably used at least in the cap.

For use in vehicle tires, the mixture as a finished mixture prior to vulcanization is brought into the corresponding shape, preferably of an outer component, and during production of the green vehicle tire is applied in the known manner.

The production of the rubber mixture of the invention for use as any other body mixture in vehicle tires is effected as described above. The difference lies in the shaping after the extrusion operation/the calendering of the mixture. The shapes thus obtained of the as-yet unvulcanized rubber mixture for one or more different body mixtures then serve for the construction of a green tire.

“Body mixture” refers here to the rubber mixtures for the inner components of a tire, such as essentially squeegee, inner liner (inner layer), core profile, belt, shoulder, belt profile, carcass, bead reinforcement, bead profile, flange profile and bandage.

The as-yet unvulcanized green tire is subsequently vulcanized.

For use of the rubber mixture of the invention in drive belts and other belts, especially in conveyor belts, the extruded, as-yet unvulcanized mixture is brought into the appropriate shape and often provided at the same time or subsequently with strength members, for example synthetic fibers or steel cords. This usually affords a multi-ply construction consisting of one and/or more plies of rubber mixture, one and/or more plies of identical and/or different strength members and one and/or more further plies of the same and/or another rubber mixture.

The present invention further provides a vehicle tire comprising the rubber mixture of the invention containing the compound of the invention in at least one component.

The vulcanized vehicle tire, in at least one component, comprises a vulcanizate of at least one rubber mixture of the invention. It is known to those skilled in the art that most substances, for example the rubbers present, are present or may be present in chemically modified form either already after mixing or only after vulcanization.

In the context of the present invention, “vehicle tires” mean pneumatic vehicle tires and all-rubber tires, including tires for industrial and construction site vehicles, truck, car and two-wheeler tires.

It is preferable when the vehicle tire of the invention comprises the rubber mixture of the invention in at least one outer component, wherein the outer component is preferably a tread, a sidewall and/or a flange profile.

The vehicle tire according to the invention may accordingly also contain the rubber mixture of the invention containing the inventive compound of formula I) in two or more components, optionally with an adjusted composition.

A further aspect of the present invention is a process for producing the compound of formula I) which comprises at least the following process steps:
a1) providing the substances of formula A1) and A2):

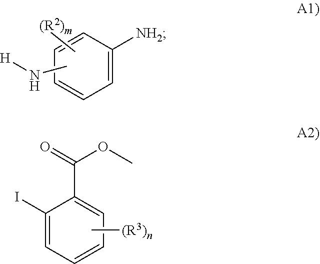

b1) converting the substances from step a1) in the presence of a base, especially an inorganic base which is especially selected from the group consisting of carbonates, such as potassium carbonate (K₂CO₃), sodium carbonate (Na₂CO₃), and phosphates, such as potassium phosphate (K₃PO₄), more preferably potassium carbonate,
and of a catalyst, especially a catalyst based on copper or palladium, where the catalyst is preferably selected from the group consisting of copper (Cu), especially copper powder, copper halides, especially copper iodide (CuI), copper bromide (CuBr), copper chloride (CuCl), and palladium complexes, more preferably copper iodide, to give the substance of formula B1):

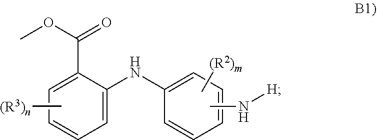

c1) reacting the compound of formula B1) with hydrogen and a ketone or aldehyde, preferably ketone, to give a compound of formula C1):

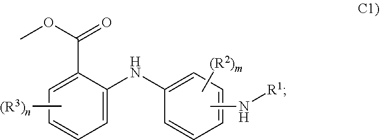

d1) providing the substances R⁴Li or R⁴Mg and R⁵Li or R⁵Mg, preferably R⁴Li and R⁵Li,
where R⁴ and R⁵ are preferably selected from the group consisting of linear, branched and cyclic aliphatic C₁ to C₁₂ radicals where, in the case that R⁴ and R⁵ are the same, R⁴Li and R⁵Li or R⁴Mg and R⁵Mg may be the same substance;
e1) reacting the substance from step c1) with the substance(s) from step d1) to give the compound of formula E1):

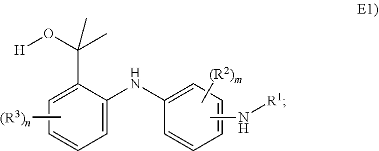

f1) reacting the substance from step e1) with a Lewis acid, preferably selected from the group consisting of boron Lewis acids such as, in particular, boron trifluoride diethyletherate, methanesulfonic acid, polyphosphoric acid, sulfuric acid, hydrochloric acid, phosphonic acid, trifluoroacetic acid, a mixture of 1,2-bis(ethenyl)benzene and 2-ethenylbenzenesulfonic acid, especially obtainable under the Amberlyst™ 15 trade name, hydrogen bromide (HBr) and para-toluenesulfonic acid, more preferably boron trifluoride diethyletherate, to give a compound of formula I):

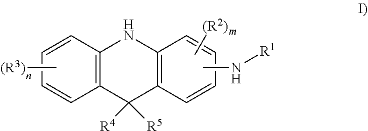

where R¹ is selected from the group consisting of benzyl and linear, branched and cyclic aliphatic C₃ to C₁₂ radicals; and
where R³ is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, and where n assumes the value of 0 or 1 or 2 or 3 or 4, where the R³ radicals, in the case that n=2 or 3 or 4, are independently the same or different, and
where R² is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals; and
where m assumes the value of 0 or 1 or 2 or 3, where the R² radicals, in the case that m=2 or 3, are independently the same or different, and where the R⁴ and R⁵ radicals are independently the same or different and are each selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, where the R⁴ and R⁵ radicals are independently the same or different and are each preferably selected from the group consisting of linear, branched and cyclic aliphatic C₁ to C₁₂ radicals, where the linear, branched and cyclic aliphatic C₁ to C₁₂ radicals and aryl radicals may bear substituents.

It is preferable when, in addition, a suitable catalyst is used in the reaction with hydrogen, referred to in the context of the present invention as “hydrogenation catalyst”.

It is preferable when the hydrogenation catalyst is a noble metal catalyst, such as in particular palladium (Pd) or platinum (Pt). It is preferable when the noble metal is used on carbon (C), such as palladium on carbon (Pd/C).

It is further also possible to use other known catalysts, such as Raney nickel or copper chromite.

More preferably, the conversion in step c1) is effected with hydrogen using a hydrogenation catalyst.

The reaction in step c1) is preferably effected at a temperature of 40 to 100° C., in particular for example 60° C.

It is preferable when hydrogen is injected to a pressure of 10 to 30 bar, in particular for example 20 bar, preferably followed by stirring for 1 to 20 hours, preferably 3 to 13 hours, more preferably 5 to 13 hours, in particular for example 10 hours.

The reaction with hydrogen in step c1) is preferably effected in a vessel suitable for the preferably comparatively high pressure, such as in particular in an autoclave or another pressure reactor.

More preferably, the reaction in step c1) with hydrogen is effected using a hydrogenation catalyst and at a temperature of 40° C. to 100° C. and wherein hydrogen is injected to a pressure of 10 to 30 bar and the reaction takes place in an autoclave or another pressure reactor.

The ketone in step c1) is the ketone derivative of the later R¹ radical; in the case of an aldehyde it is accordingly the aldehyde derivative.

For the sake of simplicity, the abbreviated formula R¹═O is used for the aldehyde or ketone since the radical R¹ is the part that remains on the nitrogen atom after the reaction with the aldehyde or ketone.

The ketone used here is preferably methyl isobutyl ketone.

The solvent in step c1) may be either the ketone or aldehyde, if this is in liquid form, or an inert solvent, such as toluene or xylene, especially if the ketone or aldehyde is in solid form. In the latter case, the ketone or aldehyde is only used as a reactant in stoichiometric quantities.

It is preferable to use a ketone or aldehyde, more preferably ketone, in liquid form as a solvent. This makes it possible to dispense with any additional substance, such as toluene or xylene.

Step c1) is preferably followed by a purification, for example by column chromatography, for example on silica gel or by recrystallization from cyclohexane or longer-chain aliphatics.

An alternative process for preparing the inventive compound of formula I) where the R⁴ and R⁵ radicals are the same and are each methyl radicals has at least the following process steps according to schemes X1) and X2):
X1)

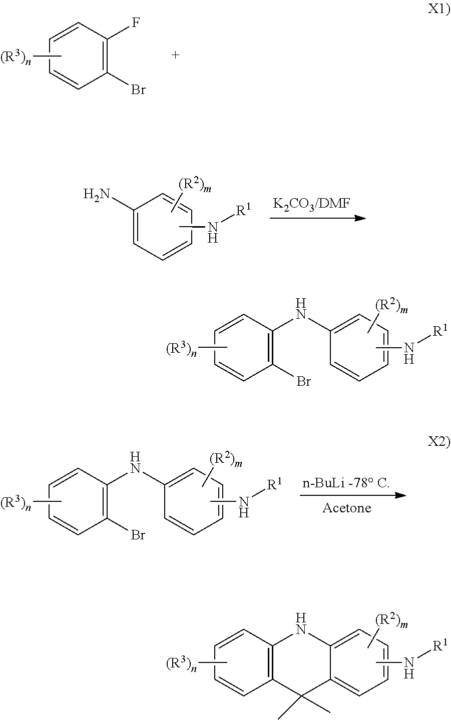

All the remarks above are applicable to the R¹, R², and R³ radicals and the indices m and n.

Step X1) is especially effected according to the disclosure of CN111269250 A1.

Step X2) is especially effected according to the disclosure of WO 2014017844 A1.

A further alternative process for preparing the inventive compound of formula I) has at least the following process steps according to schemes Y1), Y2) and Y3):

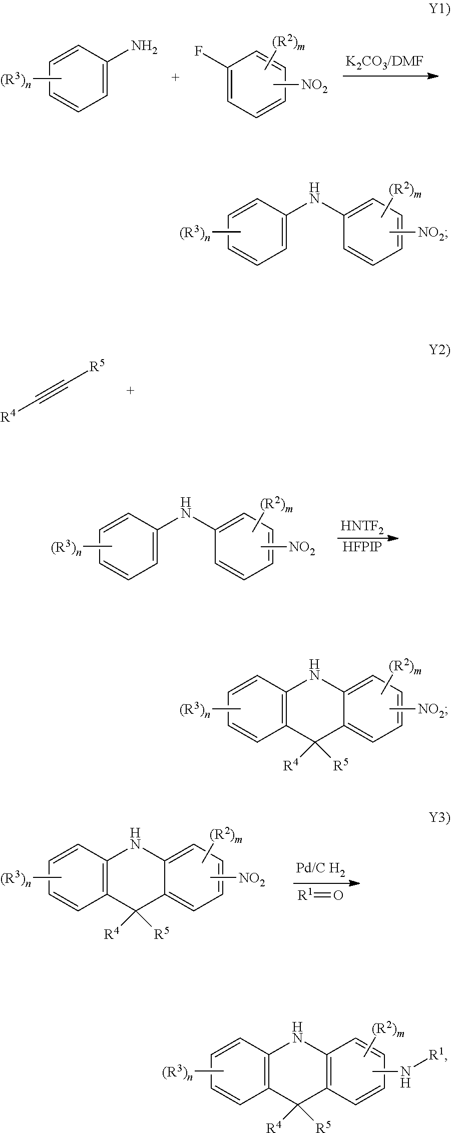

where HFPIP represents hexafluoro-2-propanol and HNTF₂ represents bis(trifluoromethane)sulfonamide, and where all the remarks above relating to formula I) are applicable to the R¹, R², R³, R⁴ and R⁵ radicals and the indices m and n.

Steps Y1) and Y2) are especially effected according to S. Wang et al., Org. Lett. 2021, 23, 7, 2565-2570.

Preferably, in step Y3), the reaction is with hydrogen and the ketone R¹=O, more preferably methyl isobutyl ketone (MIBK), under the conditions specified above for step c1).

The invention is to be elucidated in detail hereinafter by working examples.

The compound of formula II) as an illustrative embodiment of the compound of formula I) was prepared as follows according to a first synthetic route:

### Synthesis of methyl 2-(p-phenylenediamine)benzoate

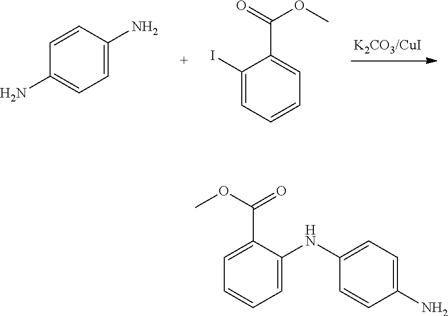 [SMILES: Nc1ccc(N)cc1 ; COC(=O)c1ccccc1I ; COC(=O)c1ccccc1Nc1ccc(N)cc1]

An initial charge was formed by 1.6 g (14.5 mmol, 2.0 eq) of p-phenylenediamine and 1.9 g of methyl 2-iodobenzoate (7.24 mmol, 1.0 eq) in 20 ml of dry dimethyl sulfoxide (DMSO). After addition of 1.00 g of potassium carbonate (K₂CO₃) (7.24 mmol, 1.0 eq) and 0.14 g of copper iodide (CuI) (0.72 mmol, 0.1 eq), the mixture was stirred at 80° C. overnight. After the reaction had ended, the solvent was removed by distillation and the residue was taken up in a mixture of ethyl acetate and 5% aqueous ammonia. Before it was dried over sodium sulfate, the organic phase was extracted by shaking once more with 5% ammonia solution, water and saturated sodium chloride solution. The inorganic salts were separated off by filtration and the solvent was removed under vacuum. The residue was purified by column chromatography on silica gel (dichloromethane (DCM)/methanol (MeOH) 95:5): An orange oil was obtained; yield 1.6 g (91% of theory).

¹H-NMR (nuclear magnetic resonator”) (500 MHZ, DMSO-d6) δ=9.00 (s, 1H), 7.83 (dd, J=8.6, 1.7 Hz, 1H), 7.29 (ddd, J=8.6, 7.0, 1.7 Hz, 1H), 6.91 (d, J=8.5 Hz, 2H), 6.79 (dd, J=8.6, 1.1 Hz, 1H), 6.67-6.56 (m, 3H), 5.07 (s, 2H), 3.84 (s, 3H).

¹³C-NMR (126 MHZ, DMSO-d6) δ=168.7, 150.4, 146.9, 134.9, 128.4, 126.6, 116.0, 115.1, 113.4, 110.0, 52.2.

### Synthesis of methyl 2-(N¹-(4-methylpentan-2-yl)-N⁴-p-phenylenediamine)benzoate

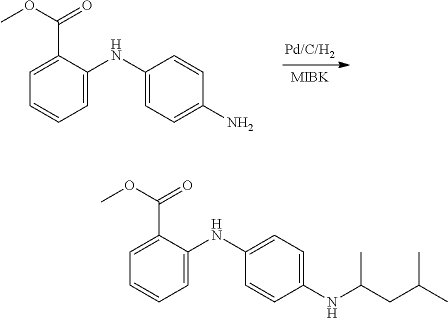 [SMILES: COC(=O)c1ccccc1Nc1ccc(N)cc1 ; COC(=O)c1ccccc1Nc1ccc(NC(C)CC(C)C)cc1]

6.80 g (28.1 mmol, 1 eq) of methyl 2-(p-phenylenediamine)benzoate, 1.18 g of palladium on carbon (Pd/C) (5%) (0.2 g on 4.67 mmol of substrate) and 50.0 ml of methyl isobutyl ketone (MIBK) were weighed into a stainless steel autoclave fitted with a Teflon liner. Hydrogen (H₂) was then injected to a pressure of 20 bar, and the mixture was left to stir at 60° C. for 10 hours. After the reaction had ended, the excess hydrogen was released and the suspension was filtered through Celite® and washed with ethanol. The filtrate was concentrated to dryness and dried under vacuum. The residue was purified by column chromatography on silica gel (cyclohexane (cyhex)/ethyl acetate (EA) 95:5). Orange oil; yield 8.20 g (89% of theory).

¹H NMR (500 MHZ, DMSO-d₆) δ=9.01 (s, 1H), 7.83 (dd, J=8.1, 1.7 Hz, 1H), 7.30 (ddd, J=8.7, 7.0, 1.7 Hz, 1H), 6.95 (d, J=8.7 Hz, 2H), 6.81 (dd, J=8.6, 1.1 Hz, 1H), 6.66-6.56 (m, 3H), 5.32 (d, J=8.6 Hz, 1H), 3.84 (s, 3H), 3.44 (dq, J=8.6, 6.7 Hz, 1H), 1.74 (dt, J=13.4, 6.7 Hz, 1H), 1.46 (dt, J=14.0, 7.1 Hz, 1H), 1.27-1.16 (m, 1H), 1.09 (d, J=6.2 Hz, 3H), 0.92 (d, J=6.6 Hz, 3H), 0.88 (d, J=6.6 Hz, 3H).

¹³C-NMR (126 MHZ, DMSO-d6) δ=168.7, 150.4, 146.6, 134.9, 131.6, 127.7, 126.8, 116.0, 113.4, 113.3, 109.9, 52.2, 46.4, 46.0, 25.00, 23.2, 23.1, 21.2.

ES-IMS (electrospray ionization mass spectrometry) [M+H]⁺=327.

### Synthesis of 2-(2-((4-((4-methylpentan-2-yl)amino)phenyl)amino)phenyl)propan-2-ol

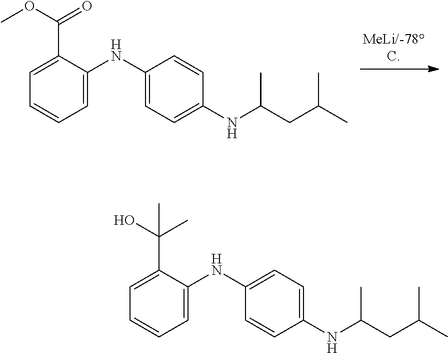 [SMILES: COC(=O)c1ccccc1Nc1ccc(NC(C)CC(C)C)cc1 ; CC(C)CC(C)Nc1ccc(Nc2ccccc2C(C)(C)O)cc1]

3.10 g of methyl 2-(N1-(4-methylpentan-2-yl)-N4-p-phenylenediamine)benzoate (9.5 mmol, 1 eq) were dissolved in 40 ml of dried diethyl ether and cooled to −78° C. Then 17.81 ml (28.5 mmol, 3 eq) of methyllithium (MeLi) were slowly added dropwise, and the mixture was stirred at −78° C. for a further 2 hours and allowed to come to room temperature (RT) overnight. The reaction was terminated by adding saturated ammonium chloride solution (NH₄Cl solution). The organic phase was extracted by shaking with water and saturated sodium chloride solution, and dried over sodium sulfate. The salts were removed by filtration, and the solvent under reduced pressure. Because of its high purity, the substance was used in the next stage without further purification: brown to black oil; yield 3.10 g (100% of theory).

¹H-NMR (500 MHZ, DMSO-d₆) δ=8.06 (s, 1H), 7.13 (dd, J=7.8, 1.6 Hz, 1H), 6.99 (ddd, J=8.5, 7.2, 1.5 Hz, 1H), 6.90-6.81 (m, 3H), 6.61 (td, J=7.4, 1.3 Hz, 1H), 6.58-6.51 (m, 2H), 5.66 (s, 1H), 4.99 (d, J=8.7 Hz, 1H), 3.40 (dq, J=7.1, 6.9 Hz, 1H), 1.74 (dh, J=14.0, 6.9 Hz, 1H), 1.45 (dt, J=13.9, 7.1 Hz, 1H), 1.21 (dt, J=13.6, 6.8 Hz, 1H), 1.08 (d, J=6.2 Hz, 3H), 0.92 (d, J=6.7 Hz, 3H), 0.88 (d, J=6.6 Hz, 3H).

¹³C-NMR (126 MHZ, DMSO) δ=145.8, 144.6, 133.0, 131.7, 127.7, 126.0, 123.6, 117.4, 114.3, 113.6, 72.9, 46.5, 46.1, 29.9, 25.0, 23.2, 23.1, 21.2.

ESI-MS [M+H]⁺=326.

### Synthesis of 2-(N¹-(4-methylpentan-2-yl)-N⁴-p-phenylenediamine)benzoic acid

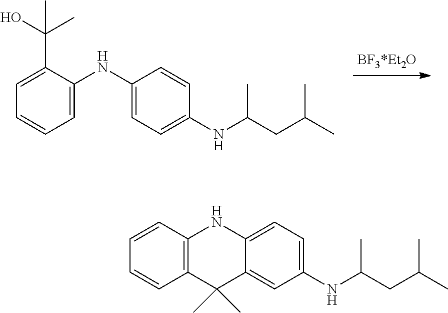 [SMILES: CC(C)CC(C)Nc1ccc(Nc2ccccc2C(C)(C)O)cc1 ; CC(C)CC(C)Nc1ccc2c(c1)C(C)(C)c1ccccc1N2]

1.00 g of 2-(2-((4-((4-methylpentan-2-yl)amino)phenyl)amino)phenyl)propan-2-ol (3.06 mmol, 1 eq) was dissolved in 40 ml of dry ether, and 1.16 ml (9.18 mmol, 3 eq) of boron trifluoride diethyletherate (BF3*Et2O) was added gradually. The reaction was stirred at RT overnight. The reaction was terminated by adding saturated NH₄Cl solution. The organic phase was extracted by shaking with saturated NaHCO₃ solution, water and saturated sodium chloride solution, and dried over sodium sulfate. After the solvent had been removed under reduced pressure, the substance was purified by chromatography (cyhex/EA 10:1). dark brown, viscous oil; yield 0.05 g (5% of theory).

¹H-NMR (500 MHZ, DMSO-d₆) δ=8.34 (s, 1H), 7.28 (dd, J=7.8, 1.4 Hz, 1H), 6.99 (ddd, J=8.3, 7.1, 1.4 Hz, 1H), 6.76-6.68 (m, 2H), 6.66-6.57 (m, 2H), 6.38 (dd, J=8.4, 2.5 Hz, 1H), 4.56 (d, J=5.7 Hz, 1H), 3.38 (br s, 1H, concealed by H₂O peak), 1.74 (dp, J=13.5, 6.7 Hz, 1H), 1.49-1.39 (m, 7H), 1.17 (dt, J=13.6, 6.9 Hz, 1H), 1.07 (d, J=6.1 Hz, 3H), 0.93 (d, J=6.6 Hz, 3H), 0.88 (d, J=6.6 Hz, 3H).

¹³C-NMR (126 MHZ, DMSO) δ=142.7, 140.4, 129.9, 129.3, 127.7, 126.7, 125.6, 118.8, 114.5, 113.4, 112.1, 110.6, 46.7, 46.6, 36.2, 31.1, 31.0, 26.8, 25.1, 23.5, 23.0, 21.3.

ESI-MS [M+H]⁺=309.

Alternatively, the compound of formula II) as an illustrative embodiment of the compound of formula I) can be prepared as follows:

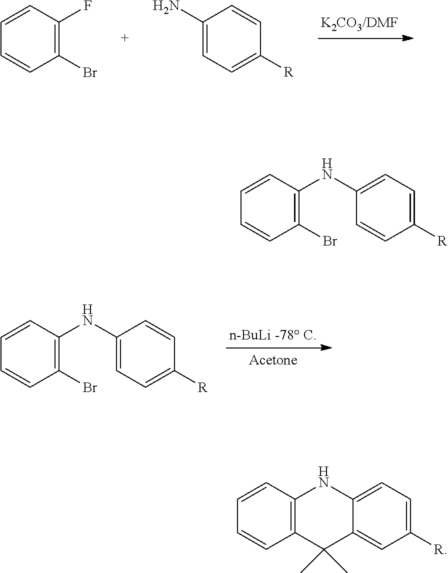

The compound of formula III) as an illustrative embodiment of the compound of formula I) can be prepared as follows:

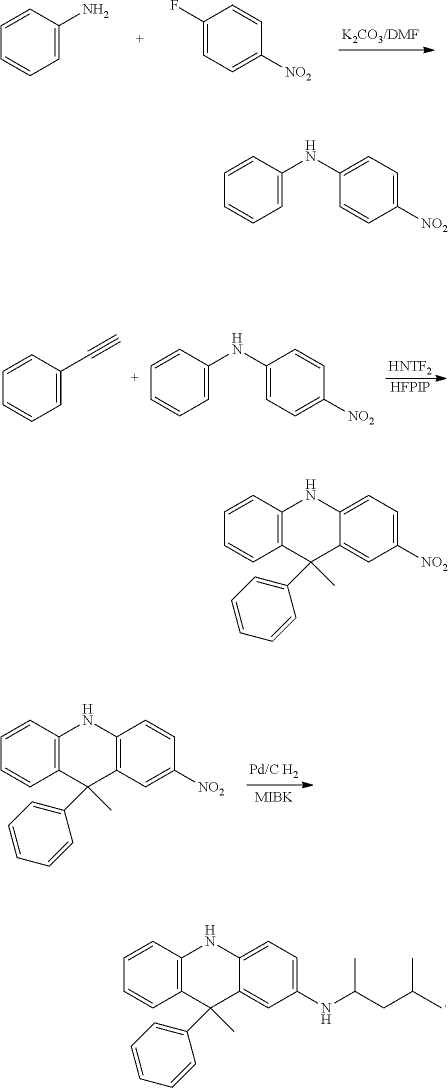

### Measurement of Oxidation Induction Time (OIT)

The compound of formula II) was investigated under laboratory conditions for its potential protective effect as an aging stabilizer by measurement of the oxidation induction time.

To this end, the compounds of formula II) and 6-PPD, each together with a polymer (liquid synthetic polyisoprene (IR), LIR-50, Kuraray, weight-average molecular weight distribution M_{w}=54 000 g/mol, glass transition temperature T_{g}=−63° C.), were heated at constant temperature (180° C.) until onset of oxidation (start temperature 35° C., heating to 170° C. at a heating rate of 20 K/min (kelvin per minute), heating to 180° C. at a heating rate of 1 K/min; purge gas: nitrogen (N₂) at a volume flow rate of 50 ml/min).

The specimen was kept under isothermal conditions at 180° C. under an N₂ atmosphere for 5 minutes and the atmosphere was then switched to an O₂ atmosphere (volume flow 50 ml/min).

Oxidation was determined via a peak using DSC (differential scanning calorimetry).

The time in minutes to oxidation was measured.

The results compared to the known aging stabilizer 6PPD are summarized in table 1.

TABLE 1

| Substance | Time [min] at 180° C. |
|---|---|
| 6PPD | 116 ± 10 |
| Formula II) | 111 ± 10 |

Taking into account the measurement accuracy of + (plus/minus) 10 minutes, it is apparent that the compound of formula II) achieves comparable protective action to 6PPD.

Even at 150° C., a comparable protective action is apparent, with termination of the test for 6PPD and for the compound of formula II) after 900 minutes. Otherwise, the procedure for the measurement was analogous to that for the measurement at 180° C., by first heating to 140° C. at a heating rate of 20 K/min (kelvin per minute) and then heating to 150° C. at a heating rate of 1 K/min.

This means that the inventive compound of formula II) as a representative of the inventive compound of formula I) is more environmentally friendly and less harmful to health than 6-PPD or other representatives of the substance class as mentioned above, and is additionally a better aging stabilizer.

With the compound of formula I), it is thus possible to achieve a comparable protective effect in the possible uses mentioned.

For use in a rubber mixture for vehicle tires, the inventive compound of formula I), for example of formula II), is added in one of the mixing stages during production of the rubber mixture in a manner known to those skilled in the art, for example instead of the aging stabilizers known in the prior art, such as 6PPD, 7PPD or IPPD, etc.

Accordingly, the compound of formula II) was incorporated in various amounts into an illustrative rubber mixture of the invention as shown in table 2. The resultant inventive examples are identified as E1 and E2.

A comparison used is rubber mixtures containing 6PPD rather than the compound of formula II) as aging stabilizer with otherwise the same composition, with replacement in each case on a mole-per-mole basis between V1 and E1, and V2 and E2. The amounts in table 2 are expressed in units of phr. In addition, a reference (Ref.) without aging stabilizer is reported.

In all mixtures, the sum total of the amounts of aging stabilizer (6PPD or formula II) and plasticizer oil MES is 10 phr.

TABLE 2

| Constituent | V1 | E1 | V2 | E2 | Ref. |
|---|---|---|---|---|---|
| NR | 100 | 100 | 100 | 100 | 100 |
| N 339 | 50 | 50 | 50 | 50 | 50 |
| carbon black | | | | | |
| MES | 8 | 7.71 | 5 | 4.26 | 10 |
| 6PPD | 2 | — | 5 | — | — |
| Compound | — | 2.29 | — | 5.74 | — |
| of formula II) | | | | | |
| ZnO | 3 | 3 | 3 | 3 | 3 |
| Stearic acid | 2 | 2 | 2 | 2 | 2 |
| TBBS | 1.2 | 1.2 | 1.2 | 1.2 | 1.2 |
| Sulfur | 1.2 | 1.2 | 1.2 | 1.2 | 1.2 |

1. A compound of formula I): 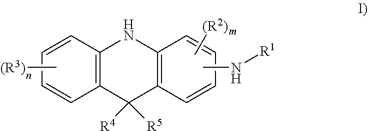
where R¹ is selected from the group consisting of benzyl and linear, branched and cyclic aliphatic C₃ to C₁₂ radicals; and
where R³ is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, and where n assumes the value of 0 or 1 or 2 or 3 or 4, where the R³ radicals, in the case that n=2 or 3 or 4, are independently the same or different, and
where R² is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals; and
where m assumes the value of 0 or 1 or 2 or 3, where the R² radicals, in the case that m=2 or 3, are independently the same or different, and
where the R⁴ and R⁵ radicals are independently the same or different and are each selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, where the linear, branched and cyclic aliphatic C₁ to C₁₂ radicals and aryl radicals may bear substituents.

2. The compound as claimed in claim 1, wherein n is 0 (zero).

3. The compound as claimed in claim, wherein m is 0 (zero).

4. The compound as claimed in claim 1, wherein R¹ is bonded to the nitrogen atom (N) via a tertiary carbon atom.

5. The compound as claimed in claim 1, wherein R¹ is a branched alkyl radical having 3 to 12 carbon atoms, preferably 3 to 8 carbon atoms.

6. The compound as claimed in claim 1, wherein R¹ is selected from 1,3-dimethylbutyl and cyclohexyl radicals, where R¹ is preferably a 1,3-dimethylbutyl radical.

7. The compound as claimed in claim 1, wherein the R⁴ and R⁵ radicals are the same and/or are each selected from the group consisting of unsubstituted, linear, branched and cyclic aliphatic C₁ to C₁₂ radicals, and aryl radicals, where the R⁴ and R⁵ radicals are more preferably selected from the group consisting of methyl radicals, n-butyl radicals and phenyl radicals, and are most preferably methyl radicals.

8. The compound as claimed in claim 1, wherein the compound has the structure of formula II):

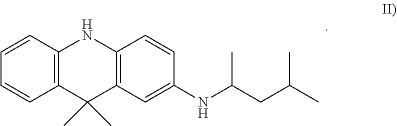 [SMILES: CC(C)CC(C)Nc1ccc2c(c1)C(C)(C)c1ccccc1N2]

9. The compound of claim 1, the compound incorporated into a rubber mixture, wherein the rubber mixture preferably contains at least one diene rubber which is particularly preferably selected from the group consisting of natural polyisoprene (NR), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

10. The compound of claim 1, the compound incorporated in at least one outer component of a tire, where the outer component is preferably a tread, a sidewall and/or a flange profile.

11. as the compound of claim 1, the compound used as an aging stabilizer and/or antiozonant especially in vehicle tires and/or other industrial rubber articles such as, in particular, an air spring, a bellows, conveyor belt, strap, drive belt, hose, rubber band, profile, a seal, a membrane, tactile sensors for medical applications or robotics applications, or a shoe sole or parts thereof, and/or oils and/or lubricants.

12. The compound of claim 1, the compound produced as a rubber article, in particular an air spring, a bellows, conveyor belt, strap, drive belt, hose, rubber band, profile, a seal, a membrane, tactile sensors for medical applications or robotics applications, or a shoe sole or parts thereof.

13. The compound of claim 1, the compound used in oils, lubricants, such as, in particular, fuels or operating media for engines.

14. The compound of claim 1, the compound incorporated as a dye in fibers and/or polymers and/or paper and/or in (decorating) paints and coatings.

15. A method for preparing a compound of formula I) which comprises the following process steps:
a1) providing the substances of formula A1) and A2): 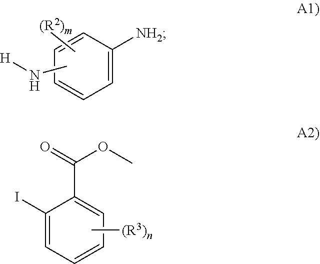
b1) converting the substances from step a1) in the presence of a base, more preferably potassium carbonate, and of a catalyst, more preferably copper iodide, to the substance of formula B1): 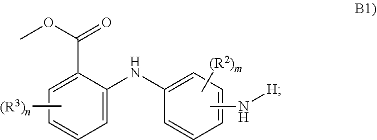
c1) reacting the compound of formula B1) with hydrogen and a ketone or aldehyde, preferably ketone, to give the compound of formula C₁): 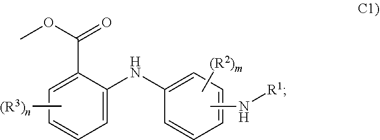
d1) providing the substances R⁴Li or R⁴Mg and R⁵Li or R⁵Mg, preferably R⁴Li and R⁵Li, where R⁴ and R⁵ are preferably selected from the group consisting of linear, branched and cyclic aliphatic C₁ to C₁₂ radicals where, in the case that R⁴ and R⁵ are the same, R⁴Li and R⁵Li or R⁴Mg and R⁵Mg may be the same substance;
e1) reacting the substance from step c1) with the substance(s) from step d1) to give the compound of formula E1): 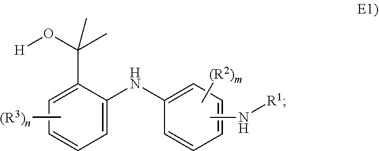
f1) reacting the substance from step e1) with a Lewis acid, preferably selected from the group consisting of boron Lewis acids such as, in particular, boron trifluoride diethyletherate, methanesulfonic acid, polyphosphoric acid, sulfuric acid, hydrochloric acid, phosphonic acid, trifluoroacetic acid, a mixture of 1,2-bis(ethenyl)benzene and 2-ethenylbenzenesulfonic acid, especially obtainable under the Amberlyst™ 15 trade name, hydrogen bromide (HBr) and para-toluenesulfonic acid, more preferably boron trifluoride diethyletherate,
to give the compound of formula I): 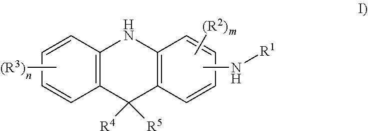
where R¹ is selected from the group consisting of benzyl and linear, branched and cyclic aliphatic C₃ to C₁₂ radicals; and
where R³ is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, and where n assumes the value of 0 or 1 or 2 or 3 or 4, where the R³ radicals, in the case that n=2 or 3 or 4, are independently the same or different, and
where R² is selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals; and
where m assumes the value of 0 or 1 or 2 or 3, where the R² radicals, in the case that m=2 or 3, are independently the same or different, and where the R⁴ and R⁵ radicals are independently the same or different and are each selected from the group consisting of linear, branched and cyclic aliphatic C₁ to C₁₂ radicals, and aryl radicals, cyano radicals, halogen radicals, preferably fluorine, bromine and chlorine, ester radicals, ketone radicals, ether radicals and thioether radicals, where the R⁴ and R⁵ radicals are independently the same or different and are each preferably selected from the group consisting of linear, branched and cyclic aliphatic C₁ to C₁₂ radicals, preferably consisting of linear C₁ to C₁₂ radicals, more preferably consisting of linear C₁ to C₆ radicals, most preferably methyl radicals, where the linear, branched and cyclic aliphatic C₁ to C₁₂ radicals and aryl radicals may bear substituents.
